(19) United States
(12) Patent Application Publication
(10) Pub. No.: US 2012/0130414 A1
(43) Pub. Date: May 24, 2012

(54) VIBRATING NEEDLE ADJUSTMENT DEVICE

(76) Inventors: Corlius Fourie Birkill, Rietondale (ZA); Paolo Amato De Risi, Menlo Park (ZA)

(21) Appl. No.: 12/950,285

(22) Filed: Nov. 19, 2010

(51) Int. Cl. A61B 17/34

(52) U.S. Cl. 606/185

(57) ABSTRACT

A vibrating needle adjustment device in accordance with an embodiment of the present application includes a housing mountable at any desired position on a needle and a vibrating element in the housing to vibrate the needle about multiple axes.

### BACKGROUND

1. Field of the Disclosure

The present disclosure relates to a vibrating needle adjustment device configured for attachment to a surgical needle to vibrate the needle about multiple axes at a desired frequency and to adjust the length of the needle.

2. Related Art

The use of needles in various surgical procedures is known. Needles are commonly used for biopsies, for injecting medication or anesthetic and for muscle or nerve stimulation. In many applications, it is important for a caregiver to be able to see or otherwise determine the position of the needle in relation to other structures in the body. For example, in the context of a biopsy needle, the caregiver preferably will be able to see the tip of the needle in the patient's body to ensure that a sample of the proper tissue is taken.

In other situations, such as delivering anesthetic to nerves, for example, it is important for the caregiver to see how close the needle is positioned to the nerve prior to injection of the anesthetic. This is equally true in the area of nerve or muscle stimulation to ensure that the needle is close enough to the nerve or muscle being stimulated while avoiding damage to the nerve or muscle.

While there are needles, which are vibrated in order to be visible on ultrasound images, these needles are generally dedicated for a particular use. Further, the vibrating mechanisms are typically component parts of a particular ultrasound system and/or require specific placement with respect to the needle to provide the desired affect. Further, these needles often only vibrate about a single axis. This may cause a problem as the needle moves through the body. At certain angles and orientations, the visibility of the needle or parts thereof may be reduced to zero. Thus, these devices are not suitable for a variety of applications and uses.

Accordingly, it would be beneficial to provide a needle adjustment device removably mounted on a needle that vibrates the needle such that it is visible on an ultrasound image.

### SUMMARY

It is an object of the present invention to provide a vibrating needle adjustment device configured for attachment to a needle at substantially any desired point and operable to vibrate the needle such that the needle is visible on an ultrasound image.

A vibrating needle adjustment device in accordance with an embodiment of the present application includes a housing including a needle path configured to receive a needle, the housing configured to be mounted on the needle at any desired point and a vibrating element mounted in the housing adjacent to the needle and configured to vibrate the needle about multiple axes.

A method of vibrating a needle in accordance with an embodiment of the present application includes mounting the vibrating needle adjustment device on the needle and vibrating the needle about multiple axes.

Other features and advantages of the present invention will become apparent from the following description of the invention which refers to the accompanying drawings.

### BRIEF DESCRIPTION OF THE DRAWINGS

FIG. 1 illustrates an embodiment of a vibrating needle adjustment device in accordance with the present application.

FIG. 2 provides a more detailed view of a vibrating needle adjustment device of FIG. 1.

FIG. 2A illustrates an embodiment of a vibrating needle adjustment device in accordance with the present application with an internal power source.

FIG. 3 illustrates a more detailed view of the vibrating element of the vibrating needle adjustment device of FIGS. 1-3.

FIG. 3A illustrates a more detailed view of the reed relay vibrating element of FIG. 3.

FIG. 3B is a graph illustrating measurements of the vibration of a needle including the vibrating needle adjustment mechanism if the present application.

FIG. 4 illustrates vibration of a needle provided using the vibrating needle adjustment device of FIGS. 1-3.

FIG. 5 illustrates an embodiment of a vibrating needle adjustment device in accordance with another embodiment of the present application.

FIG. 6 illustrates a more detailed view of the vibrating needle adjustment device of FIG. 5.

### DETAILED DESCRIPTION OF THE EMBODIMENTS

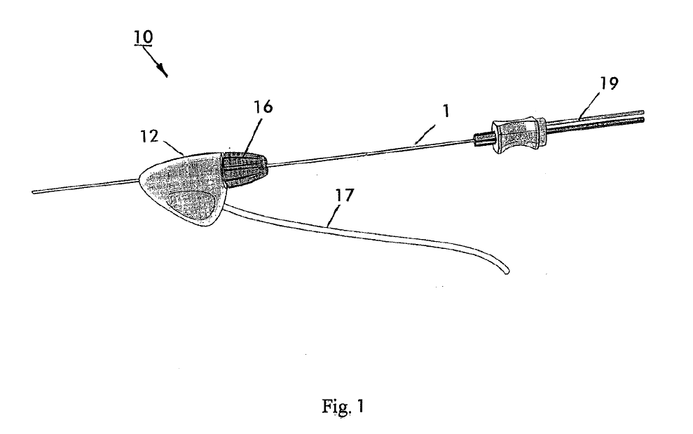

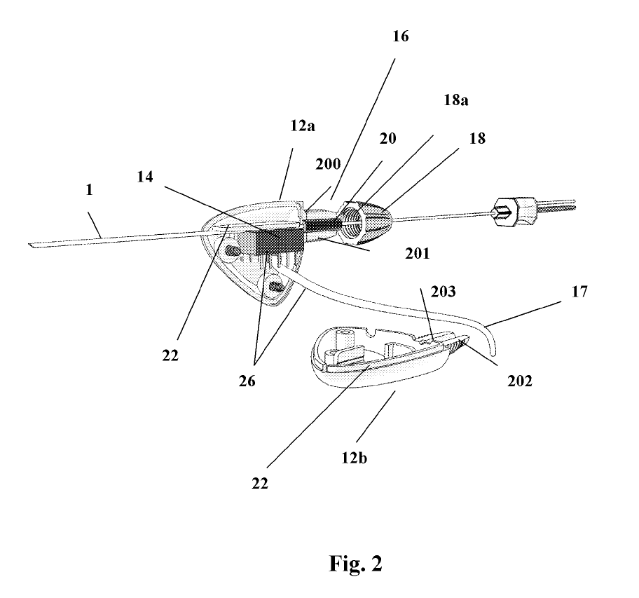

FIG. 1 illustrates an embodiment of an oscillating needle adjustment device 10 in accordance with the present disclosure. As illustrated, the device 10 preferably includes a housing 12 in which a vibrating element 14 (See FIG. 2) is mounted. The housing 12 may be positioned at substantially any desired position on a needle 1 and locked into place using locking device 16. Thus, the device 10 effectively allows the user to adjust the size of the needle to any desired length based on positioning of the housing on the needle 1. The device 10 further vibrates the needle.

FIG. 2 illustrates a more detailed view of the adjustment device 10. As illustrated, the housing 12 preferably includes two elements 12a, 12b that are connected together. In FIG. 2, screws 26 are illustrated to secure the elements 12a, 12b together, however, any suitable fastener may be used. The housing elements 12a, 12b, preferably include a needle passage 22 through which the needle 1 passes. In a preferred embodiment, the vibrating element 14 is a reed relay. The reed relay 14 is preferably mounted in the housing 12 adjacent to the needle 1.

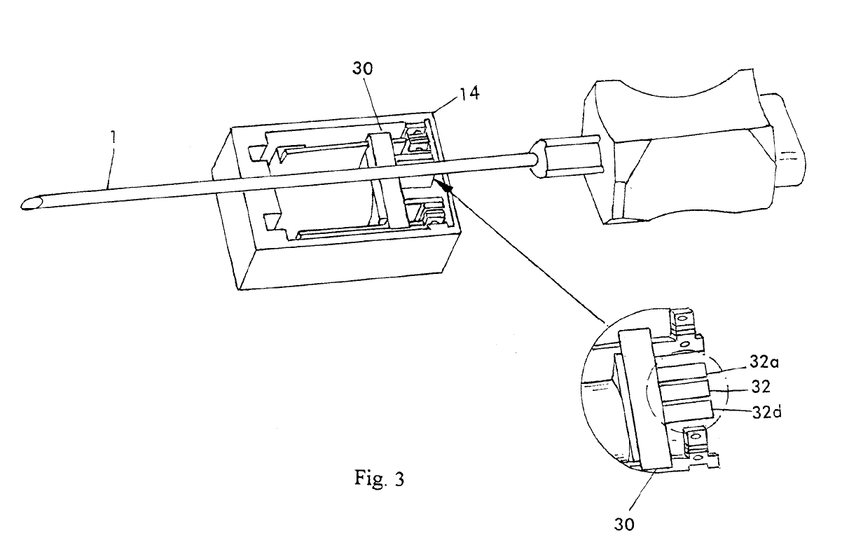

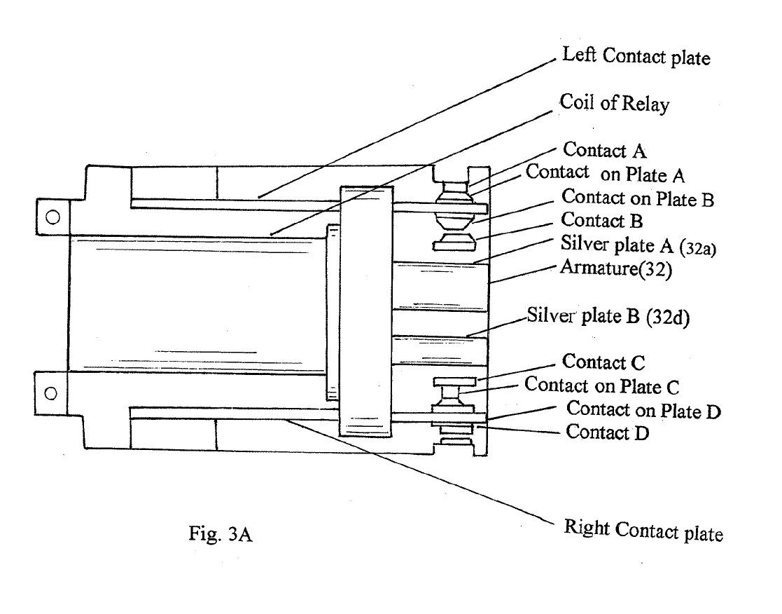

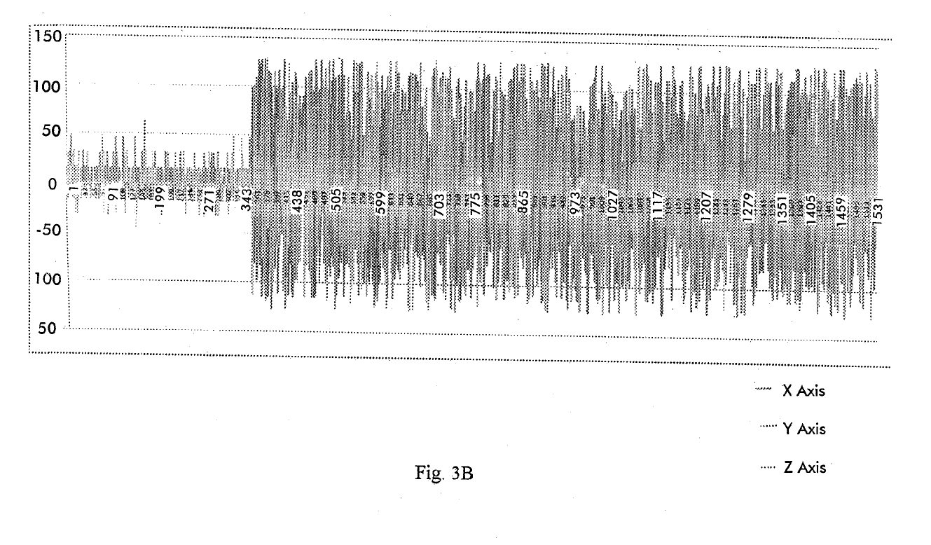

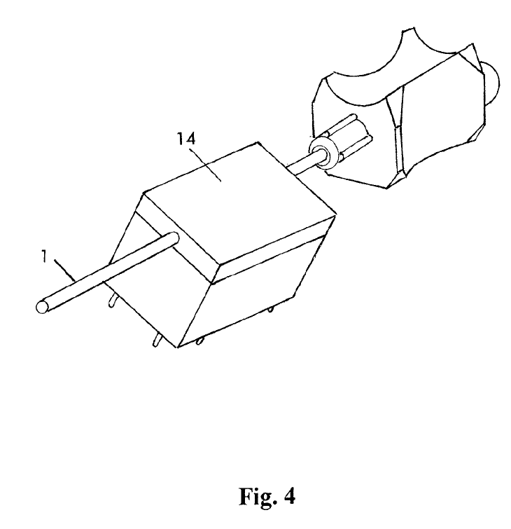

FIG. 3 illustrates a more detailed view of how the reed relay 14 operates to vibrate the needle 1. As illustrated, the needle 1 passes through the relay 14. A plastic element 30 is provided adjacent and contacting the needle 1 and connected to middle plate 32 of the relay 14. When the relay 14 is energized, the plate 32 will shift down to contact the metal plate 32d below it. The relay circuit will then de-energize and the plate 32 will return to its original position to contact the plate 32a above it. The circuit is energized in a pulsed manner at a defined frequency to provide vibrations of a desired frequency or frequency range in the needle 1. The rapid movement of plate 32 results in strong vibration of the needle 1 since the plastic element 30 moves with the plate 32 and is in direct contact with the needle 1. One advantage provided by use of the reed relay 14 is that the vibration of the needle 1 is not limited to vibration in a single axis, but tends to be in multiple axes, as illustrated in FIG. 4, for example. Vibration about multiple axes is preferred since it ensures high visibility of the needle 1 on an ultrasound image even as the angle between the ultrasound probe and the needle changes as it moves through the body.

FIG. 3A is a more detailed view of the reed relay 14. The collisions of the two metal parts, i.e. the left contact plate colliding with contacts A & B and the right contact plate colliding with contacts C&D are inelastic collisions such that the total kinetic energy is not the same before and after collision. This is evident as the collision of the plates causes sound i.e. the kinetic energy is transferred into sound and heat. The vibrations are transferred to the relay housing and to the needle.

When the relay 14 is energized, the left contact plate will swing to contact B and the right contact plate will swing to contact D. This will cause the left and right contact plates to overshoot, which will cause the original neutral (unbent) plates to bend in the opposite direction. This will cause the plates to move rapidly back and forth, back and forth with smaller and smaller deviations until the original unbent position is restored. This back and forth change in motion is vibration. When the relay is de-energized, the reverse processes will begin. The left contact plate will swing to contact A and right contact plate will swing to contact C this will cause the left and right contact plates to overshoot, which will cause the original neutral (unbent) plates to bend in the opposite direction. The plates will move rapidly back and forth, back and forth with smaller and smaller deviations until the original unbent position is restored. The back and forth change in motion is vibration. The armature (plate 32) adds more vibration as it moves from silver plate A (plate 32a) through to silver plate B (plate 32d). The net result of this vibration is to impart vibration into the needle 1 about all axes as shown in FIG. 4.

FIG. 3B is a graph representing results of measuring vibration of a needle including the device 10 of the present application using an accelerometer. On the left side of the graph, the relay was unpowered and the vibrations were the result of background noise. Once the relay is activated and oscillating the waveform shows that there is a vibration on the X, Y and Z axes, and the noise level is negligible.

The housing 12 may be locked in place on the needle 1 by tightening collet 18 of locking device 16. As can be seen in FIG. 2, the needle 1 passes through a rubber grommet 20 in the rear of the housing 12. Further, the rear of the housing 12 extends over the grommet 20 in multiple flexible sections 200, 201, 202, and 203. These flexible sections more specifically extend from the rear of the two housing elements 12a, 12b and are threaded to engage the internal threads 18a of the collet 18. The collet 18 is tapered from front to back such that as the collet 18 is screwed onto the flexible sections 201-203, the flexible sections are forced inward to press the grommet 20 against the needle 1. In this manner, the housing 12 is secured in place on the needle 1.

The wire 17 illustrated in FIGS. 1-2 may be provided to power the vibrating device 14. In an alternative and preferred embodiment, an internal power source 17a may be provided in the housing 12, as can be seen in FIG. 2A, for example. The internal power source 17a may be a battery or other portable power source that provides power to the vibration device 14. In addition, control circuitry may be provided to control the provision of power to the vibration device 14 such that the needle 1 vibrates at the desired frequency.

FIG. 2A illustrates an alternative embodiment of the device 10 where the power source 17a is internal to housing 12. In the embodiment specifically illustrated in FIG. 2A, the locking mechanism 16 has also be slightly modified. In this case, a slide switch 40 similar to that described further below is provided. This slide switch interacts with the flexible member 45 through which the needle 1 passes. In the flexed position, the needle slides easily through the member 45. When the slide switch 40 slides forward to extend the end of the member 45, the ends flex upward and lock the needle in place.

The needle 1, may be a biopsy needle, but is preferably used for muscle and/or nerve stimulation. Thus, in a preferred embodiment, the needle 1 acts as an electrode in a nerve or muscle-stimulating device, or in a pain-relieving device and may be electrically connected thereto, via wire 19, for example. In this case, control circuitry may be provided in the external device to provide the desired vibration. In addition, or in the alternative, the needle 1 may be connected to a reservoir of medication and act as a hypodermic needle.

The device 10 may be mounted on virtually any needle, regardless of the gauge of the needle, to provide vibration suitable for imaging the needle on an ultrasound system. In a preferred embodiment, the vibration provided by the device 10 is in a frequency range that provides high visibility in a color Doppler ultrasound image. Further, as noted above, vibration is preferably provided in multiple axes to ensure high visibility of the entire needle.

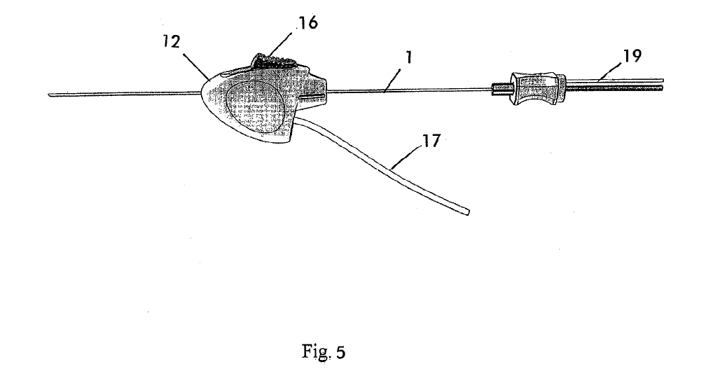

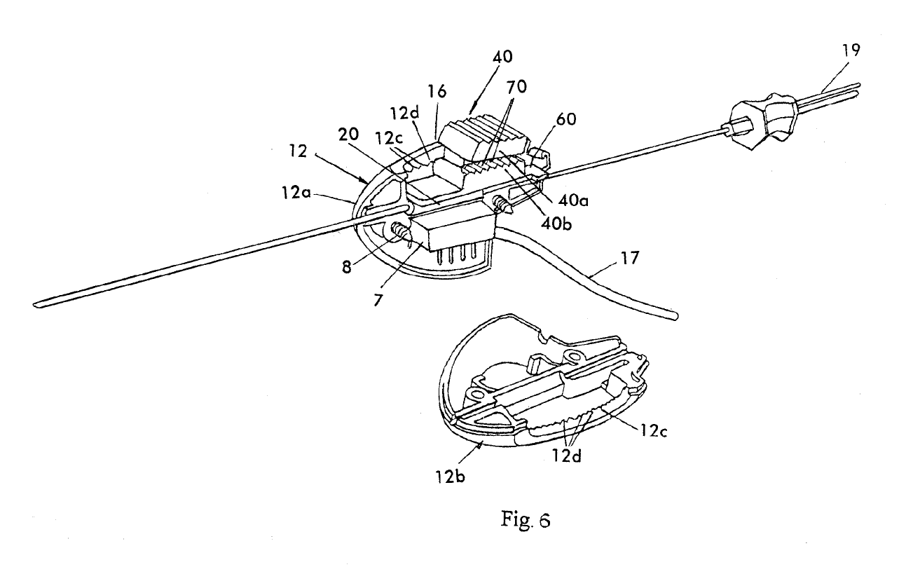

FIG. 5 illustrates an alternative embodiment of the device 10 utilizing a different locking mechanism 16 to secure the housing 12 to the needle 1. As illustrated in FIG. 6, a slide switch 40 is preferably used as the locking device 16. The slide switch 40 slides from an open position where the housing 12 is easily slidable along the needle 1 to a closed position where the housing 12 is locked into place on the needle.

As can be seen in FIG. 6, the housing 12 includes elements 12a and 12b joined together in a manner similar to that described above. A flexible plastic element 60 is provided under the slide switch 40. A grommet 20 is provided under the flexible plastic tab 60. The slide switch 40 preferably includes a cam surface 40a on a top of the base portion 40b thereof. The cam surface cooperates with a curved inner surface 12c of the housing 12 such that the switch 40 pushes the tab 60 down to increase pressure on the grommet 20 as the switch 40 slides in the direction of arrow A in FIG. 6. The increased pressure on the grommet 20 locks the housing 12 in place on the needle 1. As illustrated, a plurality of notches 70 are provided on the switch 40 to mate with notches 12d on the inner surface of the housing 12. The notches 70, 12d allow the switch 40 to be locked into a variety of positions, thus providing different levels of tension on the grommet 18. Otherwise, the device of FIGS. 5-6 is substantially similar to the device of FIGS. 1-4.

While the present application illustrates three specific examples of a locking device 16 used to secure the housing 12 in place on the needle 12, any suitable locking device 16 may be used.

Accordingly, the vibrating needle adjustment device 10 of the present disclosure may be positioned at any desired position on virtually any desired needle for use in a variety of applications. The device 10 provides for vibration of the needle in all directions, and thus, offers high needle visibility, particularly in a color Doppler ultrasound image.

Although the present invention has been described in relation to particular embodiments thereof, many other variations and modifications and other uses will become apparent to those skilled in the art.

What is claimed is:

1. A needle adjustment device comprises:
a housing configured to receive a needle, the housing configured to be mounted on the needle at any desired position along the needle; and
a vibrating element mounted in the housing adjacent to the needle and configured to vibrate the needle about multiple axes.

2. The needle adjustment device of claim 1, further comprising a locking device configured to lock the housing at a specific desired position on the needle.

3. The vibrating needle adjustment device of claim 2, wherein the vibrating element comprises a reed relay.

4. The needle adjustment device of claim 3, wherein the reed relay switches at a desired frequency to vibrate the needle at the desired frequency.

5. The needle adjustment device of claim 3, further comprising a power source configured to provide power to the reed relay.

6. The needle adjustment device of claim 5, further comprising control circuitry electrically connected to the power source and the reed relay and configured to control power delivery to the reed relay to provide vibration at the desired frequency.

7. The needle adjustment device of claim 6, wherein the power source is mounted in the housing.

8. The needle adjustment device of claim 7, wherein the control circuit is mounted in the housing.

9. The adjustment device of claim 1, wherein the housing is configured to be mounted on a needle of substantially any gauge.

10. A method of vibrating a needle utilizing a needle adjustment device comprising:
mounting the vibrating needle adjustment device on the needle at a desired position; and
vibrating the needle about multiple axes.

11. The method of vibrating a needle of claim 10, further comprising locking the vibrating needle adjustment device at the desired point on the needle.

12. The method of vibrating a needle of claim 10, wherein the vibrating step further comprises providing a vibrating element in the needle adjustment device configured to vibrate the needle.

13. The method of vibrating a needle of claim 12, wherein the vibrating step further comprises:
providing a reed relay as the vibrating element; and
switching the reed relay at a desired frequency to vibrate the needle at the desired frequency.

14. The method of vibrating a needle of claim 10, further comprising:
providing a power source; and
connecting the power source to the reed relay to provide power to the reed relay.

15. The method of vibrating a needle of claim 14, further comprising controlling the power provided to the reed relay with a control circuit to provide vibration at the desired frequency.

16. The method of vibrating a needle of claim 14, further comprising mounting the power source in the vibrating needle adjustment device.

17. The method of vibrating a needle of claim 15, further comprising mounting the control circuit in the vibrating needle adjustment device.
